SEQUENCE LISTING (3 sequences)

SEQ ID NO: 1 - DNA, 2078 bp, Bacillus stearothermophilus
  misc_structure (1)..(6): PstI Restriction Site
  misc_signal (157)..(159): Start codon
  misc_structure (157)..(489): Coding sequence for truncated sspL gene from Geobacillus stearothermophilus
  misc_structure (490)..(2072): Coding sequence for alpha-glucosidase enzyme.
  misc_signal (2070)..(2072): Stop codon
  misc_structure (2073)..(2078): BbsI Restriction Site
ctgcagaatggctgcatcaaacttcgcaaatttaacataatacatagtacagtcagcagccattgggcacagccgagaaggcagtgactccccggattcacggctggtgcaactccagcaagcccaaccatactatatataaaaggagatataacaatggctgcatcaaacttcgcaaatttaacataatacatagtacagtcagcagccattgggcacagccgagaaggcagtgactccccggattcacggctggtgcaactccagcaagcccaaccatactatatataaaaggagatataacaatgccaaaccaatctggaagtaactcttcaaaccaactacttgtacctggcgcagctcaggtaatcgatcaaatgaaattcgaaggcggcggcggcagcttgaaaaaaacatggtggaaagagggcgttgcgtatcaaatttatcctcgcagctttatggatgccaacggcgacggcatcggtgatcttcgcggcatcatcgaaaagctggattatttggtggagcttggagtcgacatcgtttggatttgtccgatttaccggtcgccgaatgccgataacggatatgacatcagcgattattatgccattatggatgagtttggaacgatggatgacttcgatgaattgcttgcccaagcccatcggcgcgggttgaaaatcattttggatttggtcatcaatcatacgagtgatgagcatccgtggtttatcgaatcgcggtcatcgcgagacaatccgaagcgcgactggtacatttggcgcgacggcaaagacgggcgcgaaccgaacaactgggaaagcattttcggcggctcggcatggcagtatgacgagcggacgggtcagtattacttgcatatttttgacgtcaaacagcccgacttgaattgggaaaacgacgaagtgcggcgggcgctttatgcgatgatcaactggtggctggataaaggcatcgacggctttcgcatcgacgcgatttcccacattaagaaaaagccgggtcttcccgatttgccaaatccgaaggggctgaagtacgtgccgtcatttgctgcgcacatgaaccagccagggattatggagtatttgcgagagttgaaagagcaaacgtttgcacgatatgacattatgacggtcggcgaggcgaacggagtaacggttgatgaggccgaacaatgggtcggggaagaaaacggcgtgtttcatatgatttttcagtttgagcatttagggctttggaaaaggaaagccgatggttcgatcgatgtccgccggctgaagcggacgttgacgaaatggcaaaaaggattggaaaaccgtgggtggaacgcgctctttttggaaaaccacgacttgcctcgatcagtgtcgacatggggaaatgacggcgagtattgggcggagagcgcgaaggcgcttggcgcgctctactttttcatgcaagggacgccgttcatttaccaagggcaagagatcgggatgacgaacgtgcaattctccgacattcgcgactaccgcgatgtcgctgccttgcgtctgtatgaactcgaacgggcgaacggccggacacatgaggaagtgatgaagatcatttggcaaaccgggcgcgacaactcgcgcaccccgatgcaatggtctggtgccccaaacgctggattcacgacaggtacgccatggatcaaggtgaacgaaaactatcgtacgatcaatgtcgaggccgagcggcgcgacccgaactcggtatggtcgttttatcgacaaatgattcagcttcggaaagcgaacgagctgtttgtttatggaacgtacgatctgcttttggaaaaccacccatccatttacgcgtacacaagaacgcttggccgcgatcgggcgcttatcattgtcaacgtatccgatcgtccttcactttaccgctatgacggcttccgccttcagtcaagcgatttggcgctctcgaactacccggtccgtccgcataaaaatgcgacgcgttttaagctgaagccgtacgaggcgcgtgtatacatctggaaagaataagaagac

SEQ ID NO: 2 - DNA, 2063 bp, Bacillus stearothermophilus
  misc_structure (1)..(6): KpnI restriction site
  misc_structure (157)..(159): Start codon
  misc_structure (157)..(391): Coding sequence for truncated sspL gene
  misc_structure (392)..(2057): Coding sequence for alpha-glucosidase gene
  misc_signal (2055)..(2057): Stop codon
  misc_structure (2058)..(2063): EcoRI restriction site
ggtaccaatggctgcatcaaacttcgcaaatttaacataatacatagtacagtcagcagccattgggcacagccgagaaggcagtgactccccggattcacggctggtgcaactccagcaagcccaaccatactatatataaaaggagatataacaatggctgcatcaaacttcgcaaatttaacataatacatagtacagtcagcagccattgggcacagccgagaaggcagtgactccccggattcacggctggtgcaactccagcaagcccaaccatactatatataaaaggagatataacaatgccaaaccaatctggaagtaactcttcaaaccaactacttgtacctggcgcagctcaggtaatcgatcaaatgaaattcgaaatgaaaaaaacatggtggaaagagggcgttgcgtatcaaatttatcctcgcagctttatggatgccaacggcgacggcatcggtgatcttcgcggcatcatcgaaaagctggattatttggtggagcttggagtcgacatcgtttggatttgtccgatttaccggtcgccgaatgccgataacggatatgacatcagcgattattatgccattatggatgagtttggaacgatggatgacttcgatgaattgcttgcccaagcccatcggcgcgggttgaaaatcattttggatttggtcatcaatcatacgagtgatgagcatccgtggtttatcgaatcgcggtcatcgcgagacaatccgaagcgcgactggtacatttggcgcgacggcaaagacgggcgcgaaccgaacaactgggaaagcattttcggcggctcggcatggcagtatgacgagcggacgggtcagtattacttgcatatttttgacgtcaaacagcccgacttgaattgggaaaacgacgaagtgcggcgggcgctttatgcgatgatcaactggtggctggataaaggcatcgacggctttcgcatcgacgcgatttcccacattaagaaaaagccgggtcttcccgatttgccaaatccgaaggggctgaagtacgtgccgtcatttgctgcgcacatgaaccagccagggattatggagtatttgcgagagttgaaagagcaaacgtttgcacgatatgacattatgacggtcggcgaggcgaacggagtaacggttgatgaggccgaacaatgggtcggggaagaaaacggcgtgtttcatatgatttttcagtttgagcatttagggctttggaaaaggaaagccgatggttcgatcgatgtccgccggctgaagcggacgttgacgaaatggcaaaaaggattggaaaaccgtgggtggaacgcgctctttttggaaaaccacgacttgcctcgatcagtgtcgacatggggaaatgacggcgagtattgggcggagagcgcgaaggcgcttggcgcgctctactttttcatgcaagggacgccgttcatttaccaagggcaagagatcgggatgacgaacgtgcaattctccgacattcgcgactaccgcgatgtcgctgccttgcgtctgtatgaactcgaacgggcgaacggccggacacatgaggaagtgatgaagatcatttggcaaaccgggcgcgacaactcgcgcaccccgatgcaatggtctggtgccccaaacgctggattcacgacaggtacgccatggatcaaggtgaacgaaaactatcgtacgatcaatgtcgaggccgagcggcgcgacccgaactcggtatggtcgttttatcgacaaatgattcagcttcggaaagcgaacgagctgtttgtttatggaacgtacgatctgcttttggaaaaccacccatccatttacgcgtacacaagaacgcttggccgcgatcgggcgcttatcattgtcaacgtatccgatcgtccttcactttaccgctatgacggcttccgccttcagtcaagcgatttggcgctctcgaactacccggtccgtccgcataaaaatgcgacgcgttttaagctgaagccgtacgaggcgcgtgtatacatctggaaagaataagaattc

SEQ ID NO: 3 - DNA, 1137 bp, Bacillus stearothermophilus
  misc_structure (1)..(6): HindIII restriction site
  misc_structure (488)..(490): Start codon for pyrF gene
  misc_structure (1131)..(1137): PstI restriction site
aagcttacgtggctaatatcacagaaattgcattagcgatcgaggaagcgggagcggacggtcttacgatgatcaacacactaatcggcatgagactcgatttaaaaaccggcaaaccgatattagcgaataaaacagggggactttcgggccctgctgtgaagccggttgccattcgcatggtgtatgaagtcagccagatggtcaacatcccgattatcggaatgggaggcgtgcaaacggctgaagatgccctggaatttcttctcgcgggagcaagcgcagtcgctgtcggaacagcaaactttgtgaatccttttgcatgtccagagattattgaacagctcccatctgttttgctccaatacggctatcaatcaattgaagaatgcatcggaaggagctggaatcatgaaaaacaacctgcccatcatcgcgcttgattttgcgtcagctgaagaaacacttgcgttcttagcgccttttcagcaagaaccgttatttgtaaaggttgggatggagcttttttatcaagaagggccatctatcgtgaaacaactaaaagaaagaaactgcgagctatttttagatctaaagcttcatgacatcccgactactgtaaacaaagcgatgaagcgccttgccagtcttggagtagacctcgtcaatgttcatgctgccgggggcaaaaaaatgatgcaggcagctctcgaaggcttagaagaaggtacgccggctggaaaaaaacgtccgtcacttatcgcggtaacccagctgacaagcacatctgaacaaatcatgaaagatgaactgctgatcgaaaagtctctgattgatacggttgtgcactacagcaaacaggcggaagaaagcggactggatggagtggtctgctctgttcatgaagcaaaagccatttaccaagcggtgtcgccttcatttctgactgtcactccggggatcagaatgtcagaggacgctgcgaatgaccaagttcgcgtagcgacgcctgccattgcaagagagaaaggttcatcagcgattgtagtaggacgctcgattacaaaagcggaagacccggtaaaagcctataaggctgtcagacttgaatgggagggaatcaaatcttgaggtacc

(19) United States
(12) Patent Application Publication
(10) Pub. No.: US 2023/0313261 A1
(43) Pub. Date: Oct. 5, 2023

(54) Biological Indicator Comprising Genetically-Modified Test Microorganism

(71) Applicant: 3M INNOVATIVE PROPERTIES COMPANY, St. Paul, MN (US)

(72) Inventors: Joshua D. Erickson, Blaine, MN (US); Narendranath Bhokisham, St. Paul, MN (US); Tonya D. Bonilla, Woodbury, MN (US); Minghua Dai, Plymouth, MN (US)

(21) Appl. No.: 18/041,842

(22) PCT Filed: Aug. 19, 2021
(86) PCT No.: PCT/IB21/57655

§ 371 Date: Feb. 16, 2023

Related U.S. Application Data
(60) Provisional application No. 63/073,543, filed on Sep. 2, 2020.

(51) Int. Cl. C12Q 1/18; C12N 9/26; A61L 2/28; A61L 2/07; C07K 14/32

(52) U.S. Cl. CPC C12Q 1/18; C12N 9/2408; C12Y 302/0102; A61L 2/28; A61L 2/07; C07K 14/32; C07K 2319/61

(57) ABSTRACT

An article for assessing efficacy of a sterilization process is provided. The article includes a housing, a plurality of genetically-modified spore-forming test microorganisms disposed in the housing, a liquid medium disposed in an openable container that is disposed in or attached to the housing, and an enzyme substrate disposed in the housing or in the openable container. The genetically-modified test microorganism comprises a functional fusion gene that encodes a non-naturally occurring chimeric protein, the chimeric protein comprising a first segment and a second segment that is contiguous with the first segment. The first segment comprises at least a portion of a first polypeptide that is normally found in spores and the second segment comprises a second polypeptide having a detectable enzymatic activity. The enzyme substrate is capable of reacting with the detectable enzyme activity to form a detectable product. A method of using the test microorganisms is also provided.

### SEQUENCE LISTING

This application contains a Sequence Listing electronically submitted via ePCT to the International Bureau as an ASCII text filed entitled “82825US002 SEQUENCE LISTING ST25.txt” having a size of 8.65 kilobytes and created on 02 September 2020. The information contained in the Sequence Listing is incorporated by reference herein.

### TECHNICAL FIELD

The present disclosure generally relates to sterilization indicators, and particularly, to biological sterilization indicators and methods of assessing the efficacy of a sterilization process.

### BACKGROUND

The sterilization of equipment, instruments, and other devices is critical in the health care industry. For example, hospitals and other medical institutions frequently sterilize medical instruments and equipment used in treating patients. The particular type of sterilization cycle used to sterilize such equipment can vary based on the particular equipment or devices being sterilized and based on the particular preference of the entity performing the sterilization cycle. However, all such sterilization cycles or processes are typically designed to kill living organisms which might otherwise contaminate the equipment or devices being sterilized.

Various sterilization methods use different cycles or techniques for sterilization. For instance, sterilization may include the administration of steam, dry heat, chemicals (e.g., ethylene oxide), or radiation, to the equipment or devices being sterilized. Steam sterilization is typically efficacious when the equipment being sterilized is heat resistant at high temperatures because the items are exposed to steam having a temperature generally in a range of 121-135° C. The period of exposure to steam depends on the sterilization temperature. For example, for equipment or instruments being sterilized are preferably exposed to the steam sterilization for approximately three minutes at 132° C. However, the exposure period could be up to 30 minutes at 121° C.

Biological indicators are commonly used to evaluate and validate the effectiveness of a sterilization process in a variety of settings. In general, viable but relatively highly-resistant spores of thermophilic organisms (test microorganisms) are subjected to the sterilization conditions along with any devices or instruments to be sterilized. In general, the spores are more resistant to the sterilization process than most other organisms that would be present by natural contamination. Applications have used spores of microorganisms capable of producing an enzyme that catalyzes the reaction of a non-fluorescent substrate to a fluorescent product that can be detected to indicate the presence of surviving spores.

Typically, after completion of the sterilization process, the test microorganisms (e.g., spores) are incubated in a liquid medium comprising an indicator compound to determine whether any of the test organisms survived the sterilization procedure. In the conventional biological indicators, a nutrient composition is brought into contact with the test microorganisms and growth of a detectable number of test microorganisms organisms can take up to 24 hours when using a pH indicator to detect the growth.

Rapid readout technology, using detection of test microorganism-associated enzyme activity, can reduce the time necessary to detect viable test microorganisms. In some implementations, an analysis of the fluorescence intensity due to a fluorescent product of an enzyme reaction serves to determine whether the sterilization process was successful.

### SUMMARY

A self-contained sterilization process biological indicator is now provided, therein with everything needed to rapidly assess the effectiveness of a variety of steam sterilization processes by enabling the detection of germination and/or outgrowth of a viable genetically-modified test microorganisms, if present, after exposing the self-contained sterilization process biological indicator to a sterilization process. Advantageously, this discovery provides its user with a biological indicator that comprises non-naturally occurring genetically-modified test microorganisms that can be detected rapidly after the sterilization process. Surprisingly, the engineered microorganisms of the present disclosure not only decrease the time needed to detect spore viability post-sterilization, but the enzyme activity of the chimeric protein is better aligned with the spore growth response after exposure to the sterilization process.

The present disclosure provides a genetically-modified test microorganism that produces a chimeric protein that comprises a detectable enzyme activity. Advantageously, the chimeric protein comprises a polypeptide that accumulates with high copy number in the spores of the test microorganism during sporulation. In addition, the fusion gene that encodes the chimeric protein increases the quantity of the enzyme activity in the spores. This feature makes it possible to detect the enzyme activity faster than it would otherwise be possible in a nongenetically-modified microorganism.

In one aspect, the present disclosure provides an article for assessing efficacy of a sterilization process. The article can comprise a housing, a plurality of genetically-modified test microorganisms disposed in the housing, a liquid medium disposed in an openable container that is disposed in or attached to the housing, and an enzyme substrate disposed in the housing or in the openable container. Each genetically-modified test microorganism of the plurality comprises a spore-forming genetically-modified test microorganism. The genetically-modified test microorganism comprises a functional fusion gene that encodes a non-naturally occurring chimeric protein, the chimeric protein comprising a first segment and a second segment that is contiguous with the first segment. The first segment can comprise at least a portion of a first polypeptide that is normally found in spores. The second segment can comprise a second polypeptide having a detectable enzymatic activity. The enzyme substrate is capable of reacting with the detectable enzyme activity to form a detectable product.

In any of the above embodiments of the article, embodiments of the self-contained sterilization process biological indicator, the first polypeptide can be selected from the group consisting of a small acid-soluble spore protein, a spore coat protein, a sporulation-associated GTP-binding protein, and spore germination proteins. In some embodiments, the first polypeptide is encoded by a gene selected from the group consisting of ysxE, yutH, cotE, cotF, cotH, cotS, cotF, gerE, sspA, sspB, sspD, and sspE. In any of the above embodiments, the second polypeptide has an enzyme activity selected from the group consisting of an esterase, a lipase, a glycosidase, an aminopeptidase, and a phosphatase, and a luciferase. In any of the above embodiments, the first segment comprises an N-terminal region of the first polypeptide. In any of the above embodiments, the at least the portion of the first polypeptide that is normally found in spores comprises a section of the first polypeptide that constitutes not less than 1% of the amino acid residues of the first polypeptide.

In another aspect, the present disclosure provides a method of determining effectiveness of a sterilization process. The method can comprise method of determining effectiveness of a sterilization process; wherein the biological indicator comprises thereon or therein a plurality of genetically-modified test microorganisms, each genetically-modified test microorganism of the plurality comprising a spore-forming genetically-modified test microorganism; wherein the genetically-modified test microorganism comprises a functional fusion gene that encodes a non-naturally occurring chimeric protein, the chimeric protein comprising a first segment and a second segment that is contiguous with the first segment; wherein the first segment comprises at least a portion of a first polypeptide that is normally found in spores; wherein the second segment comprises a second polypeptide having a detectable enzymatic activity. The method further can comprise while the indicator is positioned in the sterilization chamber, exposing the biological indicator to a sterilant gas; after exposing the biological indicator to the sterilant gas, contacting the test microorganisms with a liquid medium and an enzyme substrate capable of reacting with the enzyme activity to form a detectable product; after contacting the genetically-modified test microorganisms with the liquid medium and an enzyme substrate, incubating the biological indicator at a predetermined temperature for a period of time; and detecting the product in the liquid medium.

In some embodiments of the above method, positioning the sterilization process biological indicator in the sterilization chamber comprises positioning a sterilization process biological indicator comprising a housing with the test microorganisms therein, wherein contacting the test microorganisms with a liquid medium comprises a contacting the test microorganisms with the liquid medium inside the housing.

In yet another aspect, the present disclosure provides a kit. The kit can comprise a plurality of genetically-modified test microorganisms, each genetically-modified test microorganism of the plurality comprising a spore-forming genetically-modified test microorganism; wherein the genetically-modified test microorganism comprises a functional fusion gene that encodes a non-naturally occurring chimeric protein, the chimeric protein comprising a first segment and a second segment that is contiguous with the first segment; wherein the first segment comprises at least a portion of a first polypeptide that is normally found in spores; wherein the second segment comprises a second polypeptide having a detectable enzymatic activity. The kit further can comprise an enzyme substrate for the detectable enzyme activity.

In any embodiment, the above kit further can comprise a housing having at least one wall that forms an opening into a compartment, wherein the housing is dimensioned to contain the test microorganisms and/or the enzyme substrate. In some of the above embodiments of the kit, wherein the genetically-modified test microorganisms and/or the enzyme substrate can be disposed in the housing. In any of the above embodiments of the kit, the genetically-modified test microorganism can belong to a genus selected from the group consisting of Bacillus, Geobacillus, Clostridium, and a combination of any two or more of the foregoing genera. In any of the above embodiments of the kit, the genetically-modified test microorganisms can be mounted on a carrier. In any of the above embodiments, the kit further can comprise a liquid medium suitable for dissolving or suspending the test microorganisms and the enzyme substrate that can react with the enzyme activity of the chimeric protein to form a detectable product.

Additional details of these and other embodiments are set forth in the accompanying drawings and the description below. Other features, objects and advantages will become apparent from the description and drawings, and from the accompanying claims.

Herein, the terms “biological sterilization process indicators”, “sterilization process biological indicator”, “sterilization process indicator”, “biological indicator”, “BI”, “indicator” are used interchangeably. Likewise herein, the terms “self-contained biological indicator”, “self-contained sterilization process indicator”, “self-contained biological indicator” and “SCBI” are used interchangeably.

The term “biological sterilization process indicator”, as used herein, refers to an article that is used to evaluate the efficacy of a sterilization process and/or a piece of equipment (e.g., a sterilization chamber, an automated sterilizer, an autoclave) in which a sterilization process is conducted. The article comprises a plurality of test microorganisms (e.g., the genetically-modified test microorganisms described herein) selected for their inherent resistance to the sterilant (e.g., dry heat, moist heat, a sterilant gas) used in the sterilization process. The test microorganisms are disposed on or inside the article. Nonlimiting examples of articles that may be used as a biological sterilization process indicator include vessels such as a tube, a cuvette, a bottle, or a microwell; or a carrier substrate such as a plastic film, a paper (e.g., a filter paper), a glass slide, or a metal or ceramic coupon. “Biological sterilization process indicators” include “self-contained biological sterilization process indicators”.

A “self-contained biological sterilization process indicator”, as used herein, refers to a biological sterilization process indicator that comprises all of the components (e.g., housing, test microorganisms, liquid medium, indicator reagent(e.g., pH indictor, fluorogenic enzyme substrate) necessary to assess the survival of a genetically-modified test microorganism contained therein to determine the lethality of a sterilization process.

The numbers, E5, E6, and E7 are used interchangeably herein with 10⁵, 10⁶, and 10⁷, respectively.

The term “comprising” and variations thereof (e.g., comprises, includes, etc.) do not have a limiting meaning where these terms appear in the description and claims.

As used herein, “a”, “an”, “the,” “at least one,” and “one or more” are used interchangeably, unless the context clearly dictates otherwise.

Also herein, the recitations of numerical ranges by endpoints include all numbers subsumed within that range (e.g., 500 to 7000 includes 500, 530, 551, 575, 583, 592, 600, 620, 650, 7000, etc.).

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

The term “openable container”, as used herein, refers to a container that can be actuated, when desired, to release contents therein. The container can be actuated, for example, by fracturing the container, by dislodging or removing a plug, by actuating a valve to change it from a “closed” state to an “open” state, or by otherwise breaching at least a portion of the container.

The term “frangible container” refers to any container that can be acted upon to release its contents, for example by breaking it, puncturing it, shattering it, cutting it, etc.

The term “test microorganism”, as used herein, refers to a microorganism that is used in a method to determine the efficacy of a sterilization process. In general, test microorganisms are selected for use in the method because they are relatively resistant to the microbicidal effects of the sterilization process. The spore form of the microorganism is frequently used as a test microorganism because it is relatively highly-resistant to the sterilization process. Test microorganisms are typically derived (e.g., descendants) from naturally-occurring (i.e., occurring in nature without human modification) microorganisms including, for example, bacteria.

The term “genetically-modified test microorganism”, as used herein, refers to a test microorganism that have been genetically modified to include a recombinant gene that encodes a non-naturally occurring chimeric protein.

All scientific and technical terms used herein have meanings commonly used in the art unless otherwise specified. The definitions provided herein are to facilitate understanding of certain terms used frequently in this application and are not meant to exclude a reasonable interpretation of those terms in the context of the present disclosure.

Unless otherwise indicated, all numbers in the description and the claims expressing feature sizes, amounts, and physical properties used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviations found in their respective testing measurements.

The terms “adjacent” and “proximate” refer to the relative position of two elements, such as, for example, two layers, that are close to each other and may or may not be necessarily in contact with each other or that may have one or more layers separating the two elements as understood by the context in which “adjacent” or “proximate” appears.

The phrase “and/or” should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

The phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF DRAWINGS

FIG. 1 is a schematic genetic map of plasmid pJet1.2.

FIG. 2 is a schematic genetic map of plasmid pSTE12.

FIG. 3 is a partially-exploded cross-sectional view of a self-contained biological indicator of the present disclosure.

FIG. 4 is a cross-sectional view, along line 3-3, of the self-contained biological indicator of the FIG. 2.

FIG. 5 is an exploded perspective view of the self-contained biological indicator of the FIGS. 3-5.

### DETAILED DESCRIPTION

Before any embodiments of the present disclosure are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. It is to be understood that other embodiments may be utilized, and structural or logical changes may be made without departing from the scope of the present disclosure. Furthermore, terms such as “front,” “rear,” “top,” “bottom,” and the like are only used to describe elements as they relate to one another, but are in no way meant to recite specific orientations of the apparatus, to indicate or imply necessary or required orientations of the apparatus, or to specify how the invention described herein will be used, mounted, displayed, or positioned in use.

The present disclosure generally relates to sterilization process indicators, kits containing genetically-modified test microorganisms for use as sterilization process indicators, and methods of use thereof. A self-contained biological indicator of the present disclosure comprises all of the components necessary to assess the survival of a genetically-modified test microorganism contained therein and can be used to determine the lethality of a sterilizing process that uses, for example, moist heat (steam), ethylene oxide, hydrogen peroxide, ozone, or combinations thereof as the sterilant.

A genetically-modified test microorganism used in a method or self-contained biological indicator of the present disclosure is genetically modified to include a functional fusion gene that encodes a non-naturally occurring chimeric protein. The chimeric protein is a protein is translated from a gene that is created through the joining of portions of two or more genes that originally encoded separate proteins. Translation of resulting gene produces a single polypeptide with functional properties derived from each of the original proteins. Thus, the chimeric protein of the present disclosure comprises a first segment and a second segment that is contiguous with the first segment. The first segment includes the portion of the chimeric protein that is proximate the N-terminal amino acid of the chimeric protein. The second segment includes the portion of the chimeric protein that is proximate the C-terminal amino acid of the chimeric protein.

The first segment of the chimeric protein comprises at least a portion of a first polypeptide that is normally found in spores (e.g., the spores of a test microorganism) and may be present in relatively high quantity in the spores. There are a number of genes in spore-forming microorganisms whose expression is elevated during sporulation leading to increased levels of protein products from these genes. These genes include, for example, ysxE, yutH, cotE, cotF, cotH, cotS, cotF, gerE, sspA, sspB, sspD, and sspE. Nonlimiting examples of suitable first polypeptides encoded by such genes include a small acid-soluble spore protein, a spore coat protein, and a sporulation-associated GTP-binding protein. Advantageously, the use of a chimeric protein that is produced during sporulation ensures that the enzyme activity of the chimeric protein is present in the spores when they are exposed to the sterilization process and that there is no delay (for biosynthesis of the chimeric protein after the spore has survived the sterilization process) in detecting the enzyme activity if the spore has survived the sterilization process.

Further advantageously, because first polypeptide may comprise a substantial portion of the protein content of the spores, the enzyme activity of the chimeric protein would also be present in relatively high quantity, thus making it possible for extremely rapid detection of any spores comprising the chimeric protein that survive a sterilization process.

The portion of the first polypeptide in the chimeric protein must be sufficient to ensure that the chimeric protein partitions into the spore during sporogenesis of the genetically-modified test microorganism. In some embodiments, the portion of the first polypeptide that is normally found in spores comprises the entire first polypeptide. In some embodiments, the portion of the first polypeptide that is normally found in spores comprises less than the entire first polypeptide (e.g., at least 95%, at least 90%, at least 85%, at least 80%, at least 75%, at least 70%, at least 65%, at least 60%, at least 55%, at least 50%, at least 40%, at least 30%, at least 25%, at least 20%, at least 15%, at least 10%, at least 5%, at least 2%, or at least 1% of the first polypeptide).

The second segment of the chimeric protein comprises at least a portion of a second polypeptide that is normally found in spores having a detectable enzymatic activity. Detectable enzyme activities are well known in the art of self-contained biological indicators including, for example, self-contained biological indicators with rapid readouts (see for example, U.S. Pat. Nos. 5,223,401; 5,252,484; 6,623,955; 6, 897,059; and 6,566,090; each of which is incorporated herein by reference in its entirety. There are a number of genes in microorganisms that are translated into polypeptides having detectable enzyme activity. These genes include, for example, mall, bglH, lacZ, phoA, phoD, and pepA. Nonlimiting examples of suitable second polypeptides encoded by such genes include α-glucosidase, α-galactosidase, lipase, esterase, acid phosphatase, alkaline phosphatase, proteases, aminopeptidase, chymotrypsin, β-glucosidase, β-galactosidase, α-glucoronidase, β-glucoronidase, phosphohydrolase, α-mannosidase, β-mannosidase, a-L-fucosidase, leucine aminopeptidase, a-L-arabinofuranoside, cysteine aminopeptidase, valine aminopeptidase, β-xylosidase, α-L-iduronidase, glucanase, cellobiosidase, cellulase, α-arabinosidase, glycanase, sulfatase, butyrate, glycosidase, arabinosidase.

The portion of the second polypeptide in the chimeric protein must be sufficient to ensure that the chimeric protein retains the enzyme activity of the second polypeptide.

In some embodiments, the fusion gene further includes genetic information for a linker polypeptide that links the first polypeptide to the second polypeptide. The linker polypeptide spaces the first polypeptide apart from the second polypeptide and may thus will help both first polypeptide and the second polypeptide fold correctly in the spore, thereby retaining their respective functional activity.

The fusion gene is introduced into the test microorganism to create the genetically-modified test microorganism. Means of introducing genes into microorganisms are well known in the art. For example, the fusion gene can be integrated into a plasmid vehicle, which can be moved into the test microorganism by a process known as transformation. From there, the fusion gene can be maintained as part of an extrachromosomal replicon (e.g., the plasmid that was used as the vehicle) or the fusion gene can be integrated into the test microorganism's chromosomal replicon by in vivo recombination. In some embodiments, the integration of the fusion gene into the genome can be mediated by insertion sequences that are known in the art. In some embodiments, the plasmid vehicle can be constructed such that the fusion gene is flanked by DNA sequences from the test microorganism and the fusion gene is integrated into the genome by homologous recombination.

Plasmids are used to transfer genes into spore-forming microorganisms. Nonlimiting examples of plasmids that can be used to introduce fusion genes into spore-forming test microorganisms include pJET1.2, pNW33N, pJF751, and the Geobacillus shuttle plasmids described by Reeve et al. (“The Geobacillus Plasmid Set: A Modular Toolkit for Thermophile Engineering”, ACS Synth. Biol., 2016, vol. 5, pp 1342-1347, which is incorporated herein by reference in its entirety). Cloning vectors and techniques for transferring genes into spore-forming microorganisms are known in the art and are described, for example, in “Genetic engineering of Geobacillus spp.” (Kananavičiūtė et al., Journal of Microbiological Methods, 2015, vol. 111, pp 31-39) and in “High osmolarity improves the electro-transformation efficiency of gram-positive bacteria Bacillus subtilis and Bacillus licheniformis” (Xue et al., Journal of Microbiological Methods, 1999, vol. 111, pp 31-39), both of which are incorporated herein by reference in their entirety.

The plasmids used to construct engineered microorganisms of the present disclosure may contain one or more DNA sequences that serve as an origin of replication (ori). The plasmids may contain one or more genetic markers. The plasmids may contain a polylinker or multiple cloning site (MCS) which may be a relatively short region containing one or more restriction sites allowing the insertion of DNA fragments. The plasmids may contain one or more genes that provide a selective marker to induce the test organism to retain the plasmid. The selective marker may comprise an antibiotic resistance gene and/or or a gene with nutritional capability. The plasmids may comprise conjugative plasmids which contain tra-genes which perform the process of conjugation, the sexual transfer of plasmids to another bacterium. Transformation of test microorganisms with recombinant plasmids comprising genes capable of expressing fusion proteins can be done using techniques that are known in the art such as, for example, those described by Reeve et al. Selective pressure to maintain the plasmids in the transformant test microorganisms can be done using techniques (e.g., antibiotic selection) that are known in the art.

Turning to the drawings, FIG. 1 shows a genetic map of one embodiment of a plasmid (pJET1.2) that is used as a vector to introduce genes (e.g., genes encoding fusion proteins) into a test microorganism (e.g., Geobacillus stearothermophilus). The plasmid includes a β-lactamase gene (AmpR), a chloramphenicol acetyltransferase gene (CAT), an origin of replication (ori), as well as a variety of unique restriction sites (not shown) that are useful for cloning purposes. The unique PstI and BbsI restriction sites described in Example 1 are shown in FIG. 1.

Insertion sequences have been used to move genes into the genome of spore-forming microorganisms. Nonlimiting examples of insertion sequences that can be used to introduce fusion genes into spore-forming test microorganisms include, for example, members of the IS4 and IS21 insertion sequence families as described in U.S. Pat. No. 9,416,393, which is incorporated herein by reference in its entirety.

In certain embodiments, the fusion gene comprises regulatory elements that facilitate expression of the chimeric protein in the genetically-modified host microorganism. These regulatory elements include but are not limited to at least one of a transcription promoter, a ribosome-binding site, a translation start codon, a signal sequence, and a translation stop codon. In some embodiments, one or more of the regulatory elements are regulatory elements that are found in the natural gene that expresses the first polypeptide that is normally found in spores.

As indicated above, self-contained biological sterilization process indicators are now provided which can be used to assess the efficacy of a variety of sterilization processes including, for example, steam sterilization processes that employ temperatures at 121° C., 132° C., 134° C., or 135° C.; ethylene oxide sterilization processes; peroxide sterilization processes, and ozone sterilization processes.

Other components used in self-contained biological indicators of the present disclosure are discussed in more detail below.

Housing

For non-limiting examples of housings suitable for use in self-contained biological indicators of the present disclosure, please see U.S. Pat. Nos. 3,661,717; 5,223,401 and 6,623,955; and U.S. Patent Application Publication Nos. 2013/0302849 and 2014/0349335; each of which is incorporated herein by reference in its entirety. In general, the housing refers to a container, usually an outer container, having walls impermeable to a sterilant, where other components of the biological indicator are located. The housing may be inside a process challenge device or may be a process challenge device itself. In some embodiments, the housing may have dimensions useful to produce a flat or generally planar biological indicator. This disclosure encompasses housings of any shape and dimensions.

The housing contains at least one opening that allows flow of a sterilant to the interior of the housing (sterilant pathway). In some embodiments, the housing may comprise a body with an opening and a cap to close that opening. In some embodiments, the cap may be capable of completely sealing the housing and eliminating any fluid communication between the interior of the housing and ambiance (e.g., closing the sterilant pathway). In general, the cap has an open position in which there is an opening (e.g., a gap) between the cap and the body of the container that allows flow of liquid or gas (e.g., a sterilant) into and out of the interior of the housing. The cap also has a closed position where the opening is sealed and any fluid flow through the gap is eliminated. In other embodiments, the cap may comprise vents that allow passage of a sterilant to the interior of the housing and create an additional sterilant pathway, even if the cap is present and in the closed position. In other preferred embodiments, however, when the cap comprises vents, placing the cap in the closed position simultaneously closes: (a) the gap between the cap and the body of the container and (b) the vents present on the cap, essentially closing the sterilant pathway.

In other embodiments, the cap may lack vents and the only sterilant pathway may be through the space between the cap and the body of the housing (or through another opening or vent, if present on the body) when the cap is the open position. In some embodiments, if vents exist on the housing, they are located on the cap. In embodiments where no other opening exists besides the opening between the cap and the body of the housing, then placing the cap in the closed position completely seals off the interior of the housing, which stops the fluid communication between the interior of the housing and ambience. In those embodiments, the sterilant pathway may be sealed when the cap is in the closed position.

Genetically-Modified Test Microorganisms

Genetically-modified test microorganisms used in a method or a self-contained biological indicator of the present disclosure are spore-forming microorganisms that are particularly resistant to a given sterilization process. In certain embodiments, the methods and self-contained biological indicators of the present disclosure include a viable culture of a known species of microorganism, usually in the form of microbial spores. Spores (e.g., bacterial spores), rather than the vegetative form of the microorganisms, are used at least partly because vegetative microorganisms are known to be relatively easily killed by sterilizing processes.

The methods and self-contained biological indicator of the present disclosure includes a plurality of genetically-modified test microorganisms disposed therein (e.g., disposed in the interior of the housing). The genetically-modified test microorganisms may be of one or more species. Typically, the biological indicator contains a predetermined quantity of at least 10³, at least 10⁴, at least 10⁵, at least 10⁶, at least 10⁷, or at least 10⁸ genetically-modified test microorganisms

By way of example only, the present disclosure describes the microorganisms used in the methods and self-contained biological indicator as being “spores;” however, it should be understood that the type of microorganism (e.g., spore) used in a particular embodiment of the biological indicator is selected for being resistant to the particular sterilization process contemplated (more resistant than the microorganisms normally present on the items to be sterilized so that inactivation of the genetically-modified test microorganisms indicates a successful sterilization.). Accordingly, an embodiment of a method or self-contained biological indicator of the present disclosure for use with a particular sterilization process (or sterilant) may comprise a different genetically-modified test microorganism (e.g., a different species) than an embodiment of a method or biological indicator of the present disclosure for use with a different sterilization process (or sterilant).

In some embodiments, the genetically-modified test microorganisms can include, but are not limited to, at least one of Geobacillus stearothermophilus, Bacillus stearothermophilus, Bacillus subtilis, Bacillus atrophaeus, Bacillus megaterium, Bacillus coagulans, Clostridium sporogenes, Bacillus pumilus, or combinations thereof.

Enzyme Substrate

A self-contained biological indicator of the present disclosure comprises an enzyme substrate for the detectable enzyme activity. The enzyme activity of the chimeric protein should be one which, following a sterilization cycle which is sublethal to the genetically-modified test microorganism, remains sufficiently active to react with a suitable enzyme substrate within one hours or less, yet be inactivated or appreciably reduced in activity following a sterilization cycle that would be lethal to the genetically-modified test microorganism.

In the context of this application, an enzyme substrate comprises a substance or mixture of substances that, when acted upon by an enzyme, are converted into an enzyme-modified product. Although the preferred substrate produces a fluorescently detectable compound, in other embodiments, the product of the enzymatic action may be a luminescent or colored material. In other embodiments, however, the enzyme substrate can consist of a compound which when reacted with the enzyme, will yield a product that will react with an additional compound or composition to yield a luminescent, fluorescent, or colored material. Preferably, the substrate should not spontaneously break down or convert to a detectable product during the sterilization process or during a subsequent incubation to detect the enzyme activity. For example, in devices used to monitor steam and dry heat sterilization, the substrate must be stable at temperatures between about 20° C. and 180° C.

In general, there are two basic types of enzyme substrate that can be used in the methods and self-contained biological indicators of this disclosure. The first type of substrate can be either fluorogenic (or chromogenic), and can be given a chemical formula such as, AB. When acted upon by the enzyme, AB breaks down into the products A and B. B, for example, could be either fluorescent or colored. A specific example of a fluorogenic substrate of this type are salts of 4-methylumbelliferyl. Other fluorogenic substrates of this type include the derivatives of 4-methylumbelliferyl, 7-amido-4-methylcoumarin (7-AMC), indoxyl and fluorescein. An example of a chromogenic substrate of this type is 5-bromo-4-chloro-3-indolyl phosphate. In the presence of phosphatase, the substrate will be broken down into indigo blue and phosphate. Other chromogenic substrates of this type include derivatives of 5-bromo-4-chloro-3-indolyl, nitrophenol and phenolphthalein, listed below.

The second type of substrate can be given the chemical formula CD, for example, which will be converted by a specific enzyme into C and D. In this case, however, neither C nor D will be fluorescent or colored, but either C or D is capable of being further reacted with compound Z to give a fluorescent or colored compound, thus indicating enzyme activity. A specific fluorogenic example of this type is the amino acid lysine. In the presence of the enzyme lysine decarboxylase, lysine loses a molecule of CO₂. The remaining part of the lysine is then called cadaverine, which is strongly basic. A basic indicator such as 4-methylumbelliferone can be incorporated and will fluoresce in the presence of a strong base. A chromogenic substrate of this type would be 2-naphthyl phosphate. The enzyme phosphatase reacts with the substrate to yield beta-naphthol. The liberated beta-naphthol reacts with a chromogenic reagent containing 1-diazo-4-benzoylamino-2,5-diethoxybenzene, commercially available as “Fast Blue BB Salt” from Sigma Chemical, to produce a violet color.

As mentioned above, a preferred enzyme substrate in some embodiments is a fluorogenic substrate, defined herein as a compound capable of being enzymatically modified, e.g., by hydrolysis or other enzymatic action, to give a derivative fluorophore that has a measurably modified or increased fluorescence.

A person having ordinary skill in the art would understand that suitable fluorogenic compounds are in themselves either non-fluorescent or meta-fluorescent (i.e., fluorescent in a distinctly different way e.g., either by color or intensity, compared to the corresponding enzyme-modified products). In that regard, appropriate wavelengths of excitation and detection, in a manner known to users of fluorometric techniques, are used to separate the fluorescence signal developed by the enzyme modification from any other fluorescence that may be present.

Non-limiting examples of suitable enzymatic substrates can include, for example, derivatives of coumarin including 7-hydroxycoumarin (also known as umbelliferone or 7-hydroxy-2H-chromen-2-one) derivatives and 4-methylumbelliferone (7-hydroxy-4-methylcoumarin) derivatives including:4-methylumbelliferyl alpha-D-glucopyranoside, 4-methylumbelliferyl alpha-D-galactopyranoside, 4-methylumbelliferyl heptanoate, 4-methylumbelliferyl palmitate, 4-methylumbelliferyl oleate, 4-methylumbelliferyl acetate, 4-methylumbelliferylnonanoate, 4-methylumbelliferyl caprylate, 4-methylumbelliferyl butyrate, 4-methylumbelliferyl-beta-D-cellobioside, 4-methylumbelliferyl acetate, 4-methylumbelliferyl phosphate, 4-methylumbelliferyl sulfate, 4-methylumbelliferyl-beta-trimethylammonium cinnamate chloride, 4-methylumbelliferyl-beta-D-N, N′,N″-triacetylchitotriose, 4-methylumbelliferyl-beta-D-xyloside, 4-methylumbelliferyl-N-acetyl-beta-D-glucosaminide, 4-methylumbelliferyl-N-acetyl-alpha-D-glucosaminide, 4-methylumbelliferyl propionate, 4-methylumbelliferyl stearate, 4-methylumbelliferyl-alpha-L-arabinofuranoside, 4-methylumbelliferyl alpha-L-arabinoside; methyl umbelliferyl-beta-D-N,N′-diacetyl chitobioside, 4-methylumbelliferyl elaidate, 4-Methylumbelliferyl-alpha-D-mannopyranoside, 4-methylumbelliferyl-beta-D-mannopyranoside, 4-methylumbelliferyl-beta-D-fucoside, 4-methylumbelliferyl-alpha-L-fucoside, 4-methylumbelliferyl-beta-L-fucoside, 4-methylumbelliferyl-alpha-D-galactoside, 4-methylumbelliferyl-beta-D-galactoside, 4-trifluoromethylumbelliferyl beta-D-galactoside, 4-methylumbelliferyl-alpha-D-glucoside, 4-methylumbelliferyl-beta-D-glucoside, 4-methylumbelliferyl-7,6-sulfo-2-acetamido-2-deoxy-beta-D-glucoside, 4-methylumbelliferyl-beta-D-glucuronide, 6,8-difluor-4-methylumbelliferyl-beta-D-glucuronide, 6,8-difluoro-4-methylumbelliferyl-beta-D-galactoside, 6,8-difluoro-4-methylumbelliferyl phosphate, 6,8-difluoro-4-methylumbelliferyl beta-D-xylobioside, for example. The second substrate can also be derivatives of 7-amido-4-methylcoumarin, including: Ala-Ala-Phe-7-amido-4-methylcoumarin, Boc-Gln-Ala-Arg-7-amido-4-methylcoumarin hydrochloride, Boc-Leu-Ser-Thr-Arg-7-amido-4-methylcoumarin, Boc-Val-Pro-Arg-7-amido-4-methylcoumarin hydrochloride, D-Ala-Leu-Lys-7-amido-4-methylcoumarin, L-Alanine 7-amido-4-methylcoumarin trifluoroacetate salt, L-Methionine 7-amido-4-methylcoumarin trifluoroacetate salt, L-Tyrosine 7-amido-4-methylcoumarin, Lys-Ala-7-amido-4-methylcoumarin dihydrochloride, N-p-Tosyl-Gly-Pro-Arg 7-amido-4-methylcoumarin hydrochloride, N-Succinyl-Ala-Ala-Phe-7-amido-4-methylcoumarin, N-Succinyl-Ala-Ala-Pro-Phe-7-amido-4-methylcoumarin, N-Succinyl-Ala-Phe-Lys 7-amido-4-methylcoumarin acetate salt, N-Succinyl-Leu-Leu-Val-Tyr-7-Amido-4-methylcoumarin, D-Val-Leu-Lys 7-amido-4-methylcoumarin, Fmoc-L-glutamic acid 1-(7-amido-4-methylcoumarin), Gly-Pro-7-amido-4-methylcoumarin hydrobromide, L-Leucine-7-amido-4-methylcoumarin hydrochloride, L-Proline-7-amido-4-methylcoumarin hydrobromide; other 7-hydroxycoumarin derivatives including 3-cyano-7-hydroxycoumarin (3-cyanoumbelliferone), and 7-hydroxycoumarin-3-carboxylic acid esters such as ethyl-7-hydroxycoumarin-3-carboxylate, methyl-7-hydroxycoumarin-3-carboxylate, 3-cyano-4-methylumbelliferone, 3-(4-imidazolyl)umbelliferone; derivatives of fluorescein including: 2′,7′-Bis-(2-carboxyethyl)-5-(and-6-)carboxyfluorescein, 2′,7′-bis-(2-carboxypropyl)-5-(and-6-)-carboxyfluorescein, 5- (and 6)-carboxynaphthofluorescein, Anthofluorescein, 2′,7′-Dichlorofluorescein diacetate, 5(6)-Carboxyfluorescein, 5(6)-Carboxyfluorescein diacetate, 5-(Bromomethyl)fluorescein, 5-(Iodoacetamido)fluorescein, 5-([4,6-Dichlorotriazin-2-yl]amino)fluorescein hydrochloride, 6-Carboxyfluorescein, Eosin Y, Fluorescein diacetate 5-maleimide, Fluorescein-O′-acetic acid, O′-(Carboxymethyl)fluoresceinamide, anthofluorescein, rhodols, halogenated fluorescein; derivatives of rhodamine including: Tetramethylrhodamine, Carboxy tetramethyl-rhodamine, Carboxy-X-rhodamine, Sulforhodamine 101 and Rhodamine B; afluorescamine derivatives; derivatives of benzoxanthene dyes including: seminaphthofluorones, carboxy-seminaphthofluorones seminaphthofluoresceins, seminaphthorhodafluors; derivatives of cyanine including sulfonated pentamethine and septamethine cyanine.

The concentration of enzyme substrate present in the method or self-contained biological indicator (e.g., when dissolved and/or suspended in aqueous liquid medium in the biological indicator) depends upon the identity of the particular enzyme substrate and enzyme, the amount of enzyme-product that must be generated to be detectable, either visually or by instrument, and the amount of time that one is willing to wait in order to determine whether active enzyme is present in the reaction mixture. Preferably, the amount of enzyme substrate is sufficient to react with any residual active enzyme present, after the sterilization cycle, within about an eight-hour period of time, such that at least 10⁻⁸ molar enzyme-modified product is produced. In cases where the enzyme substrate is a 4-methylumbelliferyl derivative, the inventors have been found that its concentration in the aqueous liquid medium disclosed herein is preferably between about 10⁻⁵ and 10⁻³ molar. In some embodiments, the 4-methylumbelliferyl-α-D-glucoside can be used, for example, at a concentration of about 0.05 to about 0.5 g/L (e.g., about 0.05 g/L, about 0.06 g/L, about 0.07 g/L, about 0.08 g/L, about 0.09 g/L, about 0.1 g/L, about 0.15 g/L, about 0.2 g/L, about 0.25 g/L, about 0.3 g/L, about 0.35 g/L, about 0.4 g/L, about 0.45 g/L, about 0.5 g/L) in the aqueous mixture.

pH Indicator Dye

In any embodiment, a self-contained biological indicator of the present disclosure optionally can comprise a pH indicator dye disposed in the housing (e.g., in the compartment). In certain embodiments, the pH indicator day can be bound (e.g., with high affinity) to a pH indicator dye substrate material as described in U.S. Provisional Patent Application No. 62/990,483; filed on Mar. 17, 2020 and entitled “IMMOBILIZED PH INDICATOR FOR BIOLOGICAL INDICATOR GROWTH INDICATION”, which is incorporated herein by reference in its entirety. In any embodiment, the indicator dye may be a pH indicator suitable to detect biological activity (e.g., fermentation of a carbohydrate nutrient). The indicator dye can be selected according to criteria known in the art such as, for example, pH range, compatibility with the biological activity, and solubility. In some embodiments, a salt form of the pH indicator may be used, for example, to increase the solubility of the pH indicator in an aqueous mixture. Nonlimiting examples of suitable pH indicator dyes include, for example, thymol blue, tropeolin OO, methyl yellow, methyl orange, bromophenol blue, bromocresol green, methyl red, bromothymol blue, phenol red, chlorophenol red, neutral red, naphtholphthalein, phenolphthalein, thymolphthalein, alizarin yellow, tropeolin O, nitramine, trinitrobenzoic acid, thymol blue, bromophenol blue, tetrabromphenol blue, bromocresol green, bromocresol purple, methyl red, bromothymol blue, Congo red, and cresol red. In certain embodiments, the pH indicator dye is anionic in a solution having a pH around neutral.

In some embodiments, the pH indicator dye produces a change in color when the pH decreases, indicating growth of the genetically-modified test microorganisms. In some embodiments, the pH indicator dye is bromocresol purple. The pH indicator can be used to detect a biological activity, such as the fermentation of a nutrient (e.g., a carbohydrate disposed in the biological indicator) to acid end products (suggesting survival of the genetically-modified test microorganisms). These activities can indicate the presence or absence of a viable spore following the exposure of a biological indicator to a sterilization process, for example. The bromocresol purple can be used at a concentration of about 0.03 g/L in the aqueous mixture, for example.

The combination of bromocresol purple and 4-methylumbelliferyl-α-D-glucoside represents a preferred combination of enzymatic substrate and pH indicator dye in an article or method according to the present disclosure, but other combinations are contemplated within the scope of the present disclosure.

Self-Contained Biological Indicator

The genetically-modified test microorganisms described herein can be employed in a wide variety of biological indicators known in the art to produce a biological indicator or a self-contained biological indicator according to the present disclosure. The resulting biological indicator or self-contained biological indicator is particularly useful for assessing the effectiveness of a sterilization process (e.g., a steam sterilization process).

For example, the self-contained biological indicator of U.S. Pat. No. 3,661,717; which is incorporated herein by reference in its entirety; could be modified to use the genetically-modified test microorganisms described herein. In certain embodiments, the genetically-modified test microorganisms could be provided on a carrier substrate or on an inner surface of the housing of the biological indicator.

In addition, the self-contained biological indicators of U.S. Pat. Nos. 5,223,401 and 6,623,955; which are both incorporated herein by reference in their entirety; could be modified to use the genetically-modified test microorganisms described herein. In certain embodiments, the genetically-modified test microorganisms could be provided on a carrier substrate or on an inner surface of the housing of the biological indicator.

Furthermore, the self-contained biological indicator of U.S. Patent Application Publication No. US 2013/0302849; which is incorporated herein by reference in its entirety; could be modified to use the genetically-modified test microorganisms described herein. In certain embodiments, the genetically-modified test microorganisms could be provided on a carrier substrate or on an inner surface of the housing of the biological indicator.

A person having ordinary skill in the art will recognize how other existing biological indicators could be modified with the genetically-modified test microorganisms of the present disclosure to arrive at the articles and methods of the present disclosure.

In this disclosure, the process of bringing the spores and medium together is referred to as “activation” of the self-contained biological indicator. That is, the term “activation” and variations thereof, when used with respect to a self-contained biological indicator refer generally to bringing one or more genetically-modified test microorganisms (e.g., spores) in fluid communication with the liquid medium (e.g., an aqueous liquid medium comprising a nutrient and/or an enzyme substrate). For example, when an openable container within the self-contained biological indicator that contains the liquid medium is at least partially opened (e.g., fractured, punctured, pierced, crushed, cracked, breaking, or the like), such that the medium has been put in fluid communication with the genetically-modified test microorganisms, the self-contained biological indicator can be described as having been “activated.”

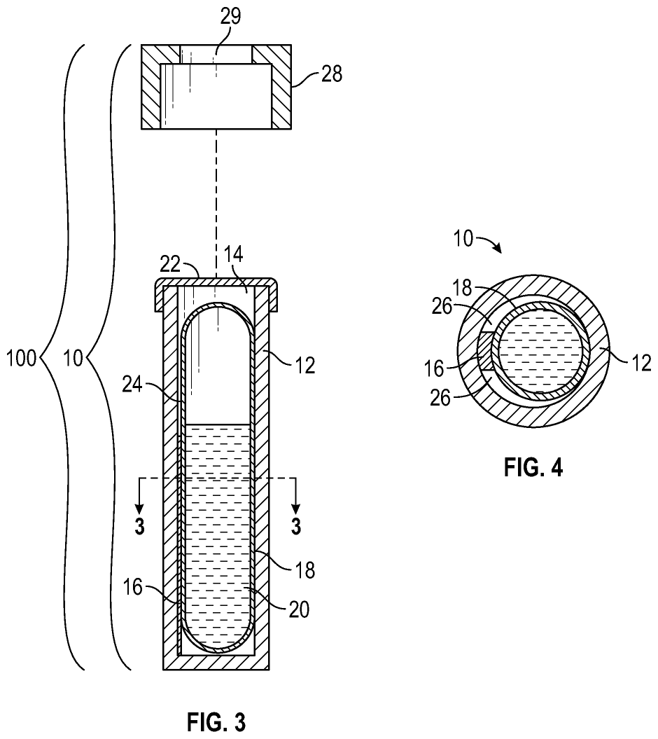

Turning now to the drawings, FIGS. 3-5 show various views of one embodiment of a self-contained biological indicator 100 according to the present disclosure.

The self-contained biological indicator 100 is shown as having a housing 10 that comprises a compartment 11 and a cap 28. The compartment 11 has at least one wall 12 that forms an opening 14. The at least one wall can be made of a moisture-impermeable, nonabsorptive material such as glass or plastic, for example. In certain preferred embodiments, the compartment is formed from an optically transparent or translucent material.

The self-contained biological indicator 100 contains a plurality of genetically-modified test microorganisms (e.g., bacterial spores) 17 disposed in the housing 10. The genetically-modified test microorganisms 17 optionally can be disposed on a carrier 16 (e.g., a sheet-like material such as a strip of filter paper or polymeric film) as a substantially water-free coating, for example. In certain embodiments, the carrier 16 is made from a water-impermeable material.

The self-contained biological indicator 100 includes a liquid medium 20 (e.g., an aqueous liquid medium) disposed in an openable container 18. The contents (e.g., liquid medium 20) of the openable container 18 are in selective communication with the compartment 11 of the housing 10. In the illustrated embodiment of FIGS. 3-5, the openable container 18 is a normally sealed, pressure-openable container, such as a frangible glass ampoule. A person having ordinary skill in the art will recognize other suitable openable containers 18 (including some embodiments wherein the container is disposed outside the housing 10) and opening means (e.g., valves, burstable seals; not shown) of selective fluid communication are disposed between the openable container and the compartment.

Enzyme Substrate

The self-contained biological indicator 100 includes an enzyme substrate capable of reacting with the detectable enzyme activity to form a detectable product. The enzyme substrate (not shown in FIGS. 3-5) is disposed in the container or the housing. In some embodiments, the enzyme substrate is dissolved and/or suspended in the liquid medium 20 that is disposed in the openable container 18 disposed in the housing 10. In certain alternative embodiments (not shown), the enzyme substrate can be disposed in the housing in a dry form (e.g., a dry coating, a powder, a tablet) that can be dissolved and/or suspended in the liquid medium upon actuation of the openable container. Additionally, upon actuation of the openable container, the liquid medium and the enzyme substrate can come into contact with the genetically-modified test microorganisms, thereby facilitating the detection of any detectable product formed by viable genetically-modified test microorganisms, if present, in the self-contained biological indicator after it has been exposed to a sterilization process.

In the illustrated embodiment of FIGS. 3-5, the container 18 is snugly retained within the compartment, so that very little of the volume of the compartment 10 remains unoccupied. The container 18 is separated from the wall 12 of the compartment 10 by the carrier 16, thereby providing cavities 24 and 26 between the wall 12 and the container 18. The open end 14 of the compartment 10 is provided with a gas-transmissive, bacteria-impermeable closure member which is shown as a sheet 22. The sheet 22 may be sealed to the open end 14 of the compartment 10 by e.g., heat or adhesive sealing, or by means of cap 28 (shown removed in FIG. 4) which has an aperture 29 therethrough adjacent the sheet 22. The aperture 29 permits passage of a sterilant, from outside the housing into the housing. The cap 28 can be formed from a variety of materials (e.g., metal, plastic) using processes that are well known in the art. During steam sterilization, for example, the steam permeates the sheet 22 and passes through cavities 24 and 26 to contact the genetically-modified test microorganisms 17 disposed on the carrier 16.

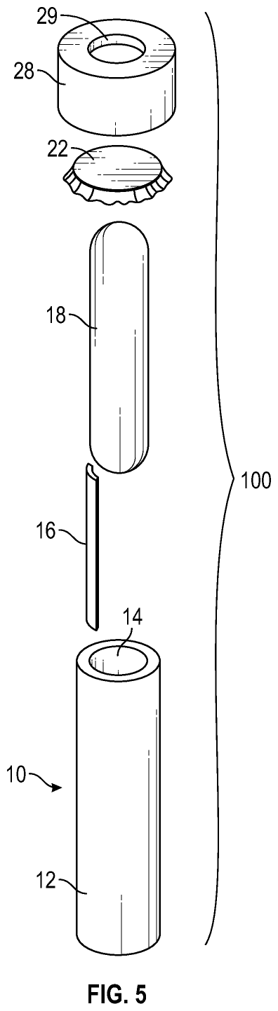

As shown in FIG. 5, the self-contained biological indicator 100 may be easily assembled by sequentially inserting into the open end 14 of the compartment 10 the carrier 16 (on which the genetically-modified test microorganisms 17 are disposed) and the container 18 and sealing the open end 14 of the tube with the sheet 22. Prior to the assembly, the genetically-modified test microorganisms 17 may be deposited onto the carrier 16 as a liquid suspension and subsequently dried before placing the carrier 16 into the container 10.

Sterilization Processes

In at least some of the sterilization processes that use steam as the sterilant, an elevated temperature, for example, 121° C., 132° C., 134° C., 135° C. or the like, is included or may be encountered in the process. In addition, elevated pressures and/or a vacuum may be encountered, for example, 15 psi (1×10⁵ Pa) at different stages within a single given sterilization cycle, or in different sterilization cycles.

In the case of steam being the sterilant, the sterilization temperatures can include 121° C., 132° C., 134° C., 135° C. The self-contained biological indicators are suitable for steam sterilization cycles at each of the temperatures above and for each temperature the cycle can have a different air removal process chosen from gravity, prevacuum (“pre-vac”), and steam flush pressure pulse (SFPP). Each of these cycles may have different exposure times depending on the type of instruments/devices being sterilized. In this disclosure, prevacuum and SFPP are also labeled as Dynamic Air Removal (DAR) cycles.

In general, a sterilization process may include placing the self-contained biological indicator of the present disclosure in a sterilizer (e.g., in the sterilization chamber of an automated sterilizer). In some embodiments, the sterilizer includes a sterilization chamber that can be sized to accommodate a plurality of articles to be sterilized and can be equipped with a means of evacuating air and/or other gases from the chamber and a means for adding steam to the chamber. The self-contained biological indicator can be positioned in areas of the sterilizer that are most difficult to sterilize. Alternately, the self-contained biological indicator can be positioned in process challenge devices to simulate sterilization conditions where steam may not be delivered as directly as would be the case in more favorable sterilization circumstances.

The steam sterilant can be added to the sterilization chamber after evacuating the chamber of at least a portion of any air or other gas present in the chamber. Alternatively, steam can be added to the chamber without evacuating the chamber. A series of evacuation steps can be used to assure that the steam sterilant reaches all desired areas within the chamber and contacts all desired article(s) to be sterilized, including the self-contained biological indicator

The self-contained biological indicators are capable of determining the efficacy of one or more steam sterilization cycles chosen from the powerset of the following eleven cycles: 121 C gravity, 121 C pre-vac, 121 C SFPP, 132 C gravity, 132 C pre-vac, 132 C SFPP, 134 C pre-vac, 134 C SFPP, 135 C gravity, 135 C pre-vac, and 135 C SFPP, preferably within less than 1 hr after removing the self-contained biological indicator from the sterilizer.

Liquid Medium

A self-contained biological indicator of the present disclosure comprises a liquid medium disposed in an openable container that is disposed in or attached to the housing. Suitable openable containers include, for example, the glass ampoule 18 described in U.S. Pat. No. 3,661,717; the inner container 48 described in U.S. Pat. No. 5,223,401; the inner compartment 18 and inner containers 48 and 78 described in U.S. Pat. No. 6,623,955; and frangible container 120 described in U.S. Patent Application Publication Nos. 2013/0302849.

The liquid medium can contain one or more of the enzyme substrates mentioned herein, provided at least one of the enzyme substrates can be converted to a detectable product by the enzymatic activity of the second polypeptide of the fusion protein. In certain embodiments, the enzyme substrate is 4-methylumbelliferyl-alpha-D-glucoside (MUG). In some embodiments, the liquid medium optionally may also include a nutrient composition that facilitates germination and/or outgrowth of the genetically-modified test microorganisms. In some embodiments, the liquid medium comprises water.

Optionally, suitable nutrients may be provided in the housing initially in a dry form (e.g., powdered form, tablet form, caplet form, capsule form, a film or coating, entrapped in a bead or other carrier, another suitable shape or configuration, or a combination thereof). When combined with the liquid medium (e.g., when the self-contained biological indicator is actuated), the nutrients can contact the genetically-modified test microorganisms and facilitate growth of any viable genetically-modified test microorganisms that remain in the housing after the self-contained biological indicator has been exposed to a sterilization process.

The nutrient can include one or more sugars, including, but not limited to, glucose, fructose, dextrose, maltose, trehalose, cellobiose, or the like, or a combination thereof. Alternatively, the nutrients may include complex media, such as peptone, tryptone, phytone peptone, yeast extract, soybean casein digest, other extracts, hydrolysates, etc., or a combination thereof. In other embodiments, the nutrient comprises a combination of one or more complex media components and other specific nutrients. The nutrient can also include a salt, including, but not limited to, sodium chloride, potassium chloride, calcium chloride, or the like, or a combination thereof. In some embodiments, the nutrient can further include at least one amino acid, including, but not limited to, at least one of methionine, phenylalanine, alanine, tyrosine, and tryptophan.

As part of a self-contained biological indicator, the liquid medium; optionally comprising nutrients, an enzyme substrate, and other components; is typically present throughout the sterilization procedure but is kept separate in the openable container and not accessible to the genetically-modified test microorganisms until the self-contained biological indicator is actuated. After the sterilization process is completed and the self-contained biological indicator is used to determine the efficacy of the sterilization, the liquid medium is placed in contact with the genetically-modified test microorganisms and the nutrient resulting in a mixture. In this disclosure, placing the liquid medium in contact with the genetically-modified test microorganisms includes activating the openable container so that the liquid medium is released and contacts the genetically-modified test microorganisms. This process may include mixing of the liquid medium with the genetically-modified test microorganisms, such as manual or mechanical shaking of the housing of the self-contained biological indicator so that the liquid medium adequately mixes with the genetically-modified test microorganisms.

In this disclosure, the process of bringing the genetically-modified test microorganisms and liquid medium together is referred to as “activation” of the self-contained biological indicator. That is, the term “activation” and variations thereof, when used with respect to a self-contained biological indicator refer generally to bringing one or more genetically-modified test microorganisms (e.g., spores) into contact with the liquid medium (optionally comprising the enzyme substrate). For example, when an openable container within the self-contained biological indicator that contains the liquid medium is at least partially fractured, punctured, pierced, crushed, cracked, breaking, or the like, such that the medium has been put in fluid communication with the genetically-modified test microorganisms, the self-contained biological indicator can be described as having been “activated.” Said another way, a self-contained biological indicator has been activated when the genetically-modified test microorganisms have been contacted with the liquid medium that was previously housed separately from the genetically-modified test microorganisms.

In some preferred embodiments, the mixture resulting from mixing the liquid medium with the genetically-modified test microorganisms after activation remains isolated within the housing of the self-contained biological indicator after the sterilization cycle has been completed and no additional reagents or components are added to it during or after activation. If at least one of the genetically-modified test microorganisms is viable, then the enzyme activity of the chimeric protein can react with the enzyme substrate, which can produce a detectable compound (e.g., a fluorescently-detectable compound).

In some embodiments, the liquid medium may comprise a buffered solution such as, for example, the buffered solution described in U.S. Patent Application No. 62/964,369 entitled “SELF-CONTAINED BIOLOGICAL INDICATOR WITH SALT COMPOUND” filed on Jan. 22, 2020; which is incorporated herein by reference in its entirety. The ionic conditions of the buffered solution should be such that the enzyme and enzyme substrate, if present in the self-contained biological indicator, are not affected. In some embodiments, a buffer solution is used as part of the liquid medium, such as phosphate buffers, (e.g., phosphate buffered saline solution, potassium phosphate or potassium phosphate dibasic), tris(hydroxymethyl) aminomethane-HCl solution, or acetate buffer, or any other buffer suitable for sterilization known in the art. Buffers suitable for the present self-contained biological indicators should be compatible with fluorogenic and/or chromogenic enzyme substrates if such enzyme substrates are used as part of the self-contained biological indicator.

The concentration of enzyme substrate, if present in the liquid medium before or after activation of the self-contained biological indicator, depends upon the identity of the particular substrate and the enzyme activity of the chimeric protein; the amount of enzyme-product that must be generated to be detectable, either visually or by instrument; and the amount of time that one is willing to wait in order to determine whether active enzyme is present in the reaction mixture. Preferably, the amount of enzyme substrate is sufficient to react with any residual active enzyme activity present in the chimeric protein, after the sterilization cycle, within less than three hours or less than one hour, such that a localized concentration of at least 10⁻⁸ molar enzyme-modified product is produced. In cases where the enzyme substrate is a 4-methylumbelliferyl derivative, the inventors have found that its concentration in the aqueous buffered solution is preferably between about 10⁻⁵ and 10⁻³ molar.

In some embodiments, the self-contained biological indicator may comprise an additional indicator compound that can facilitate the detection of another metabolic activity of the genetically-modified test microorganisms (e.g., spore) (aside from an enzyme substrate that can produce a fluorescently detectable compound). This additional metabolic activity can also be an enzymatic activity. Non-limiting examples of indicator compounds include a chromogenic enzyme substrate (e.g., observable in the visible spectrum), a pH indicator, a redox indicator, a chemiluminescent enzyme substrate, a dye, and a combination of any two or more of the foregoing indicator compounds.

In some embodiments, the additional indicator is a pH indicator that produces a change in color when the pH decreases, indicating growth of the genetically-modified test microorganisms. In some embodiments, the pH indicator is bromocresol purple. The pH indicator can be used to detect a second biological activity, such as the fermentation of a carbohydrate to acid end products (suggesting survival of the genetically-modified test microorganisms) and an enzymatic biological activity such as α-D-glucosidase enzyme activity, for example. These activities can indicate the presence or absence of a viable genetically-modified test microorganism following the exposure of a self-contained biological indicator to a sterilization process, for example. The bromocresol purple can be used at a concentration of about 0.03 g/L in the aqueous mixture, for example. The 4-methylumbelliferyl-α-D-glucoside can be used, for example, at a concentration of about 0.05 to about 0.5 g/L (e.g., about 0.05 g/L, about 0.06 g/L, about 0.07 g/L, about 0.08 g/L, about 0.09 g/L, about 0.1 g/L, about 0.15 g/L, about 0.2 g/L, about 0.25 g/L, about 0.3 g/L, about 0.35 g/L, about 0.4 g/L, about 0.45 g/L, about 0.5 g/L) in the aqueous mixture.

Method of Assessing the Efficacy of a Sterilization Process

In another aspect, the present disclosure provides a method of determining effectiveness of a sterilization process. Biological indicators, including the self-contained biological indicators of the present disclosure (e.g., SCBIs comprising a strain of Geobacillus stearothermophilus as the genetically-modified test microorganism), may be used in the method to monitor the effectiveness of one or more types of sterilization procedures, including, for example, sterilization procedures that use steam (e.g., pressurized steam) as the sterilant. “Sterilization process biological indicator”, as used in reference to a method of the present disclosure, is used broadly to include an article that contains or comprises a plurality of genetically-modified test microorganisms intended to verify the efficacy of a sterilization process. Sterilization process biological indicators include, for example, a container holding a substrate (e.g., a strip, a coupon, a bead, or a yarn made of a material known in the art of biological indicators for carrying test microorganisms during a sterilization process) having genetically-modified test microorganisms of the present disclosure affixed thereto, or an article (e.g., a strip, a coupon, a bead, or a yarn) comprising the aforementioned genetically-modified test microorganisms disposed thereon or therein.

The method comprises positioning a sterilization process biological indicator in a sterilization chamber. The biological indicator comprises thereon or therein a plurality of genetically-modified test microorganisms, each genetically-modified test microorganism of the plurality comprising a spore-forming genetically-modified test microorganism. The genetically-modified test microorganism is a test microorganism that comprises a functional fusion gene that encodes a non-naturally occurring chimeric protein as described herein. The chimeric protein comprising a first segment and a second segment that is contiguous with the first segment. The first segment comprises at least a portion of a first polypeptide that is normally found in spores and the second segment comprises a second polypeptide having a detectable enzymatic activity, both as described herein.

While the indicator is positioned in the sterilization chamber, the method comprises a step of exposing the biological indicator to a sterilant gas (e.g., steam, ethylene oxide, hydrogen peroxide vapor, ozone) for a period of time. Exposing the biological indicator to the sterilant gas comprises placing the genetically-modified test microorganisms in contact (e.g., vapor contact) with the sterilant gas.

After exposing the biological indicator to the sterilant gas, the method comprises a step of contacting the genetically-modified test microorganisms with a liquid medium and an enzyme substrate capable of reacting with the enzyme activity to form a detectable product. In view of the present disclosure, a person having ordinary skill in the art will recognize a suitable enzyme substrate based upon the enzyme activity of the second polypeptide of the chimeric protein. In certain embodiments, (e.g., in a self-contained biological indicator); the genetically-modified test microorganisms, the liquid medium, and the enzyme substrate may be co-located in a housing while the biological indicator is exposed to the sterilant gas. In certain embodiments of the method , the enzyme substrate may be disposed in the housing in a dry powder that is isolated from the liquid medium (e.g., the liquid medium may be disposed in an openable container within the housing). In certain embodiments of the method, the enzyme substrate is dissolved and/or suspended in the liquid medium that is co-located in the housing with the genetically-modified test microorganisms.

In certain embodiments, contacting the genetically-modified test microorganisms with the liquid medium comprises adding the liquid medium (e.g., by pipette) to a housing or other vessel in which the genetically-modified test microorganisms are disposed. In certain embodiments, contacting the genetically-modified test microorganisms with a liquid medium comprises activating a biological indicator (e.g., by opening a frangible container containing the medium in the biological indicator) to cause contact between the medium, the enzyme substrate, and the genetically-modified test microorganisms.

In certain alternative embodiments of the method, the biological indicator may simply comprise a substrate (e.g., a plastic film or a glass slide), optionally disposed in a housing (e.g., a tube, cuvette, or a microwell). In these embodiments, the biological indicator may have to be placed into a suitable vessel (e.g., a tube, a cuvette, a microwell) into which the liquid medium is added (e.g., by pipet). Optionally, the enzyme substrate may be added separately to the vessel or it may be dissolved and/or suspended in the liquid medium that is added to the vessel.

Optionally, after the step of contacting the genetically-modified test microorganisms with a liquid medium and an enzyme substrate capable of reacting with the enzyme activity to form a detectable product, the resulting composition comprising the genetically-modified test microorganisms, the liquid medium, and the enzyme substrate can be mixed together (e.g., by manual or mechanical agitation or vortex mixing).

After contacting the genetically-modified test microorganisms with the liquid medium and the enzyme substrate, the method comprises incubating the indicator at a predetermined temperature for a period of time sufficient to detect a presence of a product of a reaction catalyzed by the enzyme activity of the chimeric protein. The predetermined temperature can be any incubation temperature (e.g., a temperature between 25° C. and 65° C.) suitable to facilitate a reaction between the enzyme activity and the enzyme substrate. The period of time can be any suitable period of time of incubation known for detecting an enzyme reaction. In certain embodiments, the specified period of time is less than 8 hours, in some embodiments, less than 1 hour, in some embodiments, less than 30 minutes, in some embodiments, less than 15 minutes, in some embodiments, less than 5 minutes, and in some embodiments, less than 1 minute. In other embodiments, the suitable incubation time for the biological indicator of this disclosure is from 10 minutes to 4 hours, or from 10 min to 1 hr, or from 10 min to 50 min, or from 10-30 min, or from 10-20 min, or from 10-25 min, or from 15 to 30 min, or from 15-25 min, or from 15-20 min.

During and/or after incubating the mixture for the period of time, the product of the enzyme reaction can be detected using procedures known in the art including visual detection and/or automated detection. Detecting a fluorescent (or colored) product of an enzyme reaction, for example, comprises directing electromagnetic radiation (e.g., radiation within the ultraviolet or visible spectrum of electromagnetic energy) into the mixture and detecting electromagnetic radiation (e.g., radiation within the ultraviolet spectrum or visible spectrum of electromagnetic energy) emitted by the fluorescent product in the mixture, as described herein. In certain embodiments, detecting electromagnetic radiation emitted by the fluorescent product comprises detecting electromagnetic radiation using an automated detector (e.g., an auto-reader as described herein).

In any embodiment of the method, detecting a fluorescent (or colored) product of an enzyme reaction comprises detecting a quantity of fluorescence (or color) emitted (or absorbed) by the product of the enzyme reaction. In any embodiment, the quantity of fluorescence (or color) detected can be compared to a threshold quantity. In any embodiment, a first quantity of fluorescence (or color) detected after a first specified time period can be compared to a second quantity of fluorescence (or color) detected after a second specified time period. In certain embodiments, detecting at least a threshold quantity of the product of the enzyme reaction indicates a lack of efficacy of the sterilization process.

In any embodiment of the method, positioning a sterilization process biological indicator in a sterilization chamber can comprise positioning a sterilization process biological indicator comprising a housing with the genetically-modified test microorganisms therein. In these embodiments, contacting the genetically-modified test microorganisms with a liquid medium comprises a contacting the genetically-modified test microorganisms with the liquid medium inside the housing. In these embodiments, contacting the genetically-modified test microorganisms with a liquid medium can comprise contacting the genetically-modified test microorganisms with an aqueous liquid medium comprising a detection reagent (e.g., a fluorogenic or chromogenic enzyme substrate that can react with the enzyme activity of the chimeric protein).

In any embodiment of the method, detecting the product comprises measuring a parameter associated with the product to obtain a first value. To detect a detectable change caused by a viable genetically-modified test microorganism, the biological indicator can be assayed immediately after the liquid medium and the genetically-modified test microorganisms have been combined to obtain the first value (e.g., a baseline reading). After that, a second value showing any detectable change from the baseline reading can be detected. The biological indicator can be monitored and measured continuously or intermittently to observe first and second values. In some embodiments, a portion of, or the entire, incubating step may be carried out prior to measuring the detectable change.

In any embodiment of the method, measuring a parameter comprises measuring emission or absorbance of electromagnetic radiation.

In some embodiments, incubating the biological indicator at a predetermined temperature comprises incubating the biological indicator at a temperature between room temperature (e.g., about 23 degrees Centigrade) and 70 degrees Centigrade. In some embodiments, incubation can be carried out at one temperature (e.g., at 37° C., at 50-60° C., etc.), and measuring of the detectable change can be carried out at a different temperature (e.g., at room temperature, 25° C., or at 37° C.). In other embodiments, the incubation and measurement of fluorescence (or color) of the product of the enzyme reaction occurs at the same temperature.

The readout time of the biological indicator (i.e., the time to determine the effectiveness of the sterilization process) can be, in some embodiments, less than 8 hours, in some embodiments, less than 1 hour, in some embodiments, less than 30 minutes, in some embodiments, less than 15 minutes, in some embodiments, less than 5 minutes, and in some embodiments, less than 1 minute. In other embodiments, the readout time for the biological indicator of this disclosure is from 10 min to 1 hr, or from 10 min to 50 min, or from 10-30 min, or from 10-20 min, or from 10-25 min, or from 15 to 30 min, or from 15-25 min, or from 15-20 min. The detection of fluorescence (or color) above the baseline reading that would indicate presence of viable spores (i.e., a failed sterilization process) can be performed according to any method know in the art, including area under curve (in a plot of time vs fluorescence (or color) intensity), monitoring a change in slope of the curve, using a threshold value for the fluorescence (or color), etc., or a combination thereof of two or more techniques.

Kits

In another aspect, the present disclosure provides a kit that can be used for determining the efficacy of a sterilization process. The kit comprises a plurality of genetically-modified test microorganisms, each genetically-modified test microorganism of the plurality comprising a spore-forming genetically-modified test microorganism; wherein the genetically-modified test microorganism comprises a functional fusion gene that encodes a non-naturally occurring chimeric protein, the chimeric protein comprising a first segment and a second segment that is contiguous with the first segment; wherein the first segment comprises at least a portion of a first polypeptide that is normally found in spores; wherein the second segment comprises a second polypeptide having a detectable enzymatic activity. The kit further comprises an enzyme substrate for the detectable enzyme activity. In any embodiment, the kit further comprises instructions for using the genetically-modified test microorganisms to assess the efficacy of a sterilization process.

In any embodiment, the kit further comprises comprising a housing having at least one wall that forms an opening into a compartment as described hereinabove. In any embodiment, the housing is dimensioned to contain the test microorganisms and/or the enzyme substrate. In any embodiment, the genetically-modified test microorganisms and/or the enzyme substrate is disposed in the housing.

In any embodiment of the kit, the genetically-modified test microorganism belongs to a genus selected from the group consisting of Bacillus, Geobacillus, Clostridium, and a combination of any two or more of the foregoing genera. In any embodiment, the kit further comprises a liquid medium suitable for dissolving or suspending the test microorganisms and the enzyme substrate that can react with the enzyme activity of the chimeric protein to form a detectable product.

The present invention is illustrated by the following examples. It is to be understood that the particular examples, materials, amounts, and procedures are to be interpreted broadly in accordance with the scope and spirit of the invention as set forth herein.

### EXAMPLES

### Example 1. Preparation of Self-Contained Biological Indicators

Construction of a shuttle vector containing gene that expresses a fusion protein. Geobacillus stearothermophilus (ATCC 7953) cells are propagated at 60° C. in trypticase soy broth (TSB) or on trypticase soy agar (TSA). Escherichia coli (DH5a) is propagated at 3TC in Luria broth (LB). Plasmid 35 pJet1.2 is an E. coli/bacillus shuttle vector that is obtained from Addgene.org (Watertown, MA. Plasmid pJet1.2 contains a genetic marker for Ampicillin selection in pJet1.2-transformed E. coli and a genetic marker for Chloramphenicol resistance in pJet1.2-transformed Bacillus species. See FIG. 1 for annotated genetic map of pJet1.2.

The fusion gene construct shown in SEQ ID NO. 1 is synthesized by Genescript (Piscataway, NJ). This construct includes the small acid soluble protein (SASP) gene sspL from G. stearothermophilus. It also includes the promoter region of the sspL gene from the G. stearothermophilus genome. The construct is formed by fusing a portion of the N-terminus of the sspL gene in-frame with the alpha-glucosidase gene from G. stearothermophilus. A linker peptide coding region is disposed between the sspL coding portion and the alpha-glucosidase coding portion of the fusion gene construct. PstI and BbsI restriction enzyme sites are added to the N-terminus and C-terminus of the construct, respectively, and are used to insert the fusion gene into plasmid pJet1.2.

The fusion gene construct and the pJet1.2 plasmid are digested with PstI and BbsI according to restriction enzyme suppliers protocol. The digested molecules are were then ligated together causing insertion of the fusion gene construct into the PstI and BbsI sites in pJet1.2. The resulting plasmid is then transformed into E. coli DH5a, replicated and the DNA is purified with a plasmid mini-prep kit. The DNA of the purified plasmid is sequenced to ensure it contains the fusion insert and then the plasmid is transformed first into B. subtilis and then the plasmid is re-purified again from B. subtilis and transformed into G. stearothermophilus. Transformants (genetically-modified test microorganisms) are selected on TSA agar containing chloramphenicol. Chloramphenicol resistant colonies are then picked to obtain the G. stearothermophilus genetically-modified test microorganism.

Construction of biological indicators. G. stearothermophilus genetically-modified test microorganism spores are produced with chloramphenicol added to the agar sporulation media. The spores are harvested form the agar plates and then purified by washing several times with sterile deionized H₂0. The spores are then coated onto polypropylene film. The coated carriers are dried, and dye cut into individual spore carriers (ca. 2 mm×4 mm).

3M™ ATTEST™ Super Rapid Readout Biological Indicators (part number 1492V) are obtained from 3M Company (St. Paul, MN). Articles like those biological indicators are described in U.S. Patent Application Publication No. US 2014/0349335, which is incorporated herein by reference in its entirety. The caps of the 1492V biological indicators are removed and the contents (glass ampule, insert, and the spore reservoir) are removed and reassembled likewise with the exception that the spore reservoir of the commercial biological indicators are replaced with the spore carriers coated with spores of the genetically-modified test microorganisms described above.

### Example 2. Detection of Enzymatic Activity of Chimeric Protein in Self-Contained Biological Indicators Exposed to a Steam Sterilant

Ten of the assembled self-contained biological indicators of Example 1 are exposed to 132.2° C. Pre-vacuum steam sterilization cycles in an AMSCO Model 3013 automated sterilizer (Steris Corporation; Mentor, OH). The length of exposure to 132.2-degree steam is selected so that most spores are killed in each biological indicator but at least one spore would be expected to survive in about 10% to about 90% of the biological indicators (i.e. what is known in the art as a “fractional cycle”). The biological indicators comprising the genetically-modified G. stearothermophilus spores are monitored with a biological indicator fluorescence autoreader available from 3M Company (St. Paul, MN). Fluorescence detection of alpha-glucosidase activity is observed in the biological indicators in which at least one spore has survived the exposure to the steam sterilant.

### Example 3. Preparation of Alternative Self-Contained Biological Indicators

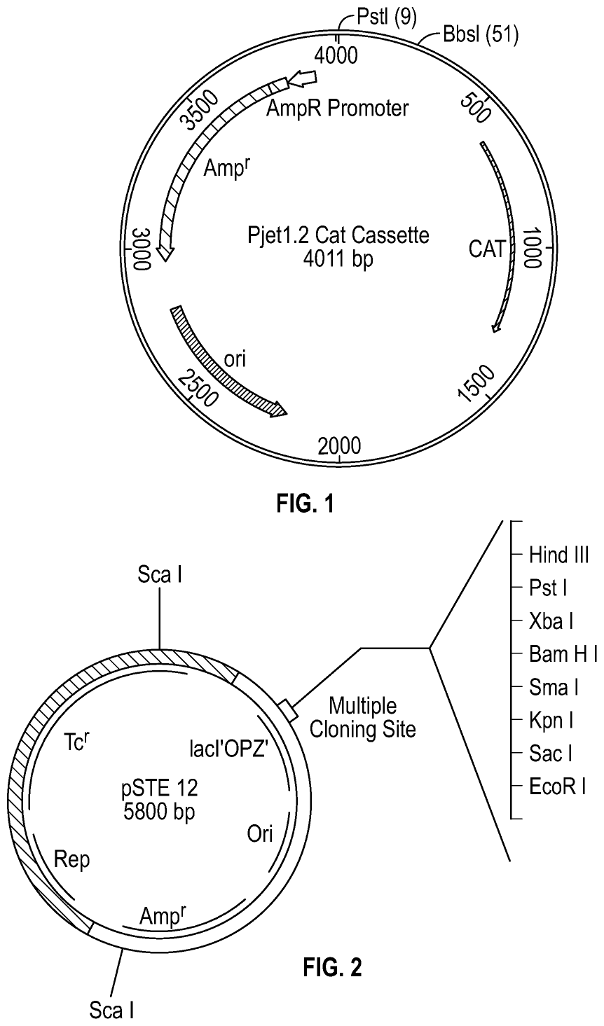

Geobacillus stearothermophilus (ATCC 7953) is propagated at 60° C. and grown in trypticase soy broth (TSB) or on trypticase soy agar (TSA). Escherichia coli (DH5a) is propagated at 3TC in Luria broth (LB). pSTE12 is an E. coli/Bacillus shuttle vector and contains a genetic marker for Ampicillin selection in E. coli and tetracycline resistance in G. stearothermophilus. pSTE12 includes ampicillin resistance (Amp′) and tetracycline resistance (Tc′) genes, an origin of replication (ori), as well as a β-galactosidase gene that includes a multiple cloning site. See FIG. 2 for annotation of pSTE12.

The fusion construct shown in SEQ ID NO. 2 is synthesized by Genescript. This construct includes the small acid soluble protein (SASP) gene sspL from G. stearothermophilus. It also includes the promoter region of the sspL gene from the G. stearothermophilus genome. The construct is formed by fusing a portion of the N-terminus of the sspL gene in-frame with the alpha-glucosidase gene from G. stearothermophilus. KpnI and EcoRI restriction enzyme sites are added to the N-terminus and C-terminus of the construct, respectively, for insertion into the multiple cloning site of pSTE12.

The pyrF gene from Bacillus spp. encodes an orotidine 5′-phosphate decarboxylase orthologue of the eukaryotic ura3 gene and is essential for pyrimidine biosynthesis and metabolization of 5-fluoroorotic acid (5-FOA) to toxic metabolites. Disruption of pyrF in Geobacillus stearothermophilus results in uracil auxotrophy and resistance to 5-FOA. The integrative plasmid is designed for single crossover homologous recombination that contains a region of homology to pyrF including upstream sequence of ˜400 bp. The pyrF gene is synthesized by Genescript and includes flanking HindIII and PstI restriction sites for insertion into the multiple cloning site of pSTE12.

The plasmid construct, comprising the fusion gene of SEQ ID NO. 2 and the pyrF gene from Geobacillus stearothermophilus is introduced into Geobacillus stearothermophilus via protoplast transformation or conjugative transfer. Transformants are selected by 5-FOA resistance in the presence of uracil and then replica plated onto agar media with or without tetracycline.

Tetracycline-resistant colonies are then selected and G. stearothermophilus spores are produced in or on sporulation media containing Tetracycline. The spores are then purified by washing several times with dH20. The spores are then coated onto polypropylene film. The coated carriers are dried, and dye cut into individual carriers (ca. 2 mm×4 mm). The carriers are then assembled into self-contained biological indicators as described in Example 1.

### Example 4. Detection of Enzymatic Activity of Chimeric Protein in Self-Contained Biological Indicators Exposed to a Steam Sterilant

The assembled BI's including the genetically engineered spores with the fusion construct are then exposed to 132.2° C. Pre-vacuum steam sterilization cycles in an AMSCO Model 3013 automated sterilizer. When compared to the same biological indicator construct with native G. stearothermophilus spores, the biological indicators with the genetically engineered spores result in more-rapid fluorescent detection with 4-methylumbeliferyl-alpha-D-glucoside (4MUG) at any given exposure time relative to the pH color change growth response.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

The complete disclosures of all patents, patent applications, publications, and nucleic acid and protein database entries which are cited herein, are hereby incorporated by reference as if individually incorporated. Various modifications and alterations of this invention will become apparent to those skilled in the art without departing from the scope and spirit of this invention, and it should be understood that this invention is not to be unduly limited to the illustrative embodiments set forth herein.

1. An article for assessing efficacy of a sterilization process, the article comprising:
a housing having at least one wall that forms an opening into a compartment;
a plurality of genetically-modified test microorganisms disposed in the housing, each genetically-modified test microorganism of the plurality comprising a spore-forming genetically-modified test microorganism;
wherein the genetically-modified test microorganism comprises a functional fusion gene that encodes a non-naturally occurring chimeric protein, the chimeric protein comprising a first segment and a second segment that is contiguous with the first segment;
wherein the first segment comprises at least a portion of a first polypeptide that is normally found in spores;
wherein the second segment comprises a second polypeptide having a detectable enzymatic activity;
a liquid medium disposed in an openable container that is disposed in or attached to the housing; and
an enzyme substrate capable of reacting with the detectable enzyme activity to form a detectable product, the enzyme substrate being disposed in the housing or in the openable container.

2. The article of claim 1, wherein the first polypeptide is selected from the group consisting of a small acid-soluble spore protein, a spore coat protein, and a sporulation-associated GTP-binding protein.

3. The article of claim 2, wherein the first polypeptide is encoded by a gene selected from the group consisting of ysxE, yutH, cotE, cotF, cotH, cotS, cotF, gerE, sspA, sspB, sspD, and sspE.

4. The article of claim 1, wherein the second polypeptide has an enzyme activity selected from the group consisting of an esterase, a lipase, a glycosidase, an aminopeptidase, and a phosphatase, and a luciferase.

5. The article of claim 4, wherein the second polypeptide has an enzyme activity selected from the group consisting of b-D-galactosidase; b-D-glucosidase; a-D-glucosidase; alkaline phosphatase; acid phosphatase; butyrate esterase; caprylate esterase; chloramphenicol acetytransferase; catechol-2,3-dioxygenase; myristate lipase; leucine aminopeptidase; valine aminopeptidase; chymotrypsin; phosphohydrolase; a-D-galactosidase; a-L-arabinofuranosidase; N-acetyl-b-glucosaminidase; b-D-cellobiosidase; alanine aminopeptidase; proline aminopeptidase; tyrosine aminopeptidase; phenylalanine aminopeptidase; b-D-glucuronidase, and fatty acid esterase.

6. The article of claim 1, wherein the first segment comprises an N-terminal region of the first polypeptide.

7. The article of claim 1, wherein the functional fusion gene includes at least one regulatory sequence that controls expression of the first polypeptide.

8. The article of claim 1, wherein the genetically-modified test microorganism belongs to a genus selected from the group consisting of Bacillus, Geobacillus, Clostridium, and a combination of any two or more of the foregoing genera.

9. The article of claim 1, wherein the liquid medium comprises an enzyme substrate dissolved and/or suspended therein.

10. The article of claim 1, wherein the at least the portion of the first polypeptide that is normally found in spores comprises a section of the first polypeptide that constitutes not less than 1% of the amino acid residues of the first polypeptide.

11. The article of claim 1, wherein the fusion gene is disposed in the genetically-modified test microorganism in an extrachromosomal replicon.

12. The article of claim 1, wherein the fusion gene is disposed in the genetically-modified test microorganism in a chromosomal replicon.

13. A method of determining effectiveness of a sterilization process, the method comprising:
positioning a sterilization process biological indicator in a sterilization chamber;
wherein the biological indicator comprises thereon or therein a plurality of genetically-modified test microorganisms, each genetically-modified test microorganism of the plurality comprising a spore-forming genetically-modified test microorganism;
wherein the genetically-modified test microorganism comprises a functional fusion gene that encodes a non-naturally occurring chimeric protein, the chimeric protein comprising a first segment and a second segment that is contiguous with the first segment;
wherein the first segment comprises at least a portion of a first polypeptide that is normally found in spores;
wherein the second segment comprises a second polypeptide having a detectable enzymatic activity;
while the indicator is positioned in the sterilization chamber, exposing the biological indicator to a sterilant gas;
after exposing the biological indicator to the sterilant gas, contacting the test microorganisms with a liquid medium and an enzyme substrate capable of reacting with the enzyme activity to form a detectable product;
after contacting the genetically-modified test microorganisms with the liquid medium and an enzyme substrate, incubating the biological indicator at a predetermined temperature for a period of time; and
detecting the product in the liquid medium.

14. The method of claim 13, wherein positioning the sterilization process biological indicator in the sterilization chamber comprises positioning a sterilization process biological indicator comprising a housing with the test microorganisms therein, wherein contacting the test microorganisms with a liquid medium comprises a contacting the test microorganisms with the liquid medium inside the housing.

15. The method of claim 14, wherein contacting the test microorganisms with the liquid medium comprises contacting the test microorganisms with an aqueous medium comprising the enzyme substrate.

16. The method of claim 13, wherein incubating the biological indicator at a predetermined temperature comprises incubating the biological indicator at a temperature between 23-70 degrees Centigrade.

17. The method of claim 13, wherein detecting the product comprises measuring a parameter associated with the product to obtain a first value.

18. The method of claim 17, wherein measuring a parameter comprises measuring emission or absorbance of electromagnetic radiation.

19. The method of claim 17, wherein the method further comprises comparing the first value to a second value.

20. A kit, comprising:
a plurality of genetically-modified test microorganisms, each genetically-modified test microorganism of the plurality comprising a spore-forming genetically-modified test microorganism;
wherein the genetically-modified test microorganism comprises a functional fusion gene that encodes a non-naturally occurring chimeric protein, the chimeric protein comprising a first segment and a second segment that is contiguous with the first segment;
wherein the first segment comprises at least a portion of a first polypeptide that is normally found in spores;
wherein the second segment comprises a second polypeptide having a detectable enzymatic activity; and
an enzyme substrate for the detectable enzyme activity.

21-26. (canceled)
